(19) United States
(12) Patent Application Publication
(10) Pub. No.: US 2022/0228223 A1
(43) Pub. Date: Jul. 21, 2022

(54) SQUAMOUS CELL CARCINOMA DIAGNOSTIC OR PROGNOSIS PREDICTION MARKER AND USE THEREOF

(71) Applicant: IUCF-HYU (INDUSTRY-UNIVERSITY COOPERATION FOUNDATION HANYANG UNIVERSITY), Seoul (KR)

(72) Inventors: Jin-Wu NAM, Seoul (KR); Bo-Hyun YOU, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 17/614,755

(22) PCT Filed: May 8, 2020
(86) PCT No.: PCT/KR2020/006063

§ 371 Date: Nov. 29, 2021

(30) Foreign Application Priority Data
May 28, 2019 (KR) 10-2019-0062423

(51) Int. Cl. C12Q 1/6886

(52) U.S. Cl. CPC C12Q 1/6886; C12Q 2600/178; C12Q 2600/118

(57) ABSTRACT

The present invention relates to a squamous cell carcinoma diagnostic or prognosis prediction marker and the use thereof and, more particularly, to the use of a long non-coding RNA as a marker composition for diagnosing or predicting the prognosis of squamous cell carcinoma, a diagnostic composition, diagnostic information-providing method and the like, by means of the correlation between an expression of the long non-coding RNA and squamous cell carcinoma pathogenesis. The inventors discovered that the expression pattern of a long non-coding RNA is related to cancer pathogenesis, investigated the correlation between the expression of a specific long non-coding RNA and squamous cell carcinoma pathogenesis and thus confirmed that the long non-coding RNA can be a squamous cell carcinoma diagnostic or prognosis prediction marker. Therefore, the marker according to the present invention can assist management and treatment of a cancer patient by means of diagnosing or predicting the prognosis of squamous cell carcinoma.

### TECHNICAL FIELD

The present invention relates to a squamous cell carcinoma diagnostic or prognosis prediction marker and the use thereof and, more particularly, to the use of a long non-coding RNA as a marker composition for diagnosing or predicting the prognosis of squamous cell carcinoma, a diagnostic composition, a diagnostic information-providing method and the like, by means of the correlation between the expression of the long non-coding RNA and squamous cell carcinoma pathogenesis.

### BACKGROUND ART

Squamous cell carcinoma is a carcinoma occurring in squamous cells such as the esophagus, head and neck, and lungs, and has been reported to have a poor prognosis due to a very high metastasis rate, and in particular, esophageal squamous cell carcinoma is known to have a 5 year survival rate of only 5 to 10%.

Meanwhile, squamous cell carcinoma is diagnosed by allowing a patient to drink a contrast medium which is impermeable to X-rays depending on the site of occurrence and confirming the contrast medium adhering to the mucosa with X-rays or by directly observing the site by endoscopy. However, since the diagnostic method can mainly observe only the cell surface, it is difficult to determine the degree or depth of cancer progression. Further, since it is difficult to feel symptoms of squamous cell carcinoma at the initial stage, it is often diagnosed in a state in which the cancer has progressed.

Therefore, in order to ensure the treatment of cancer, patients with squamous cell carcinoma receive treatment such as radiation therapy and anticancer chemotherapy in advance, and then receive treatment to remove a tissue which is highly likely to develop cancer in advance. In addition, since even a skilled doctor cannot be sure whether the above-mentioned cancer is treated before the surgical result of a patient with the squamous cell carcinoma condition is obtained, an affected part of the patient is excised as much as possible such that patients who have undergone surgery are reported to have a poor quality of life and suffer from sequelae such as complications.

Meanwhile, since squamous cell carcinoma responds differently to anticancer chemotherapy depending on the size and extent of the cancer and the patient's intrinsic factors, an accurate prognosis for each patient needs to be predicted.

Thus, although there is a patent (Korean Patent No. 10-1247636) for a marker for diagnosing squamous cell carcinoma, it is still insufficient to meet the above need, and there is a very urgent need for the initial diagnosis of the cancer and the prediction of the prognosis of a patient through non-invasive examination using a marker for diagnosing squamous cell carcinoma.

### DISCLOSURE

### Technical Problem

The inventors discovered that the expression pattern of a long non-coding RNA is related to squamous cell carcinoma pathogenesis, and confirmed that an expression of a particular long non-coding RNA could lead to a marker for diagnosing or predicting the prognosis of squamous cell carcinoma, thereby completing the present invention.

Thus, an object of the present invention is to provide a marker composition for diagnosing or predicting the prognosis of squamous cell carcinoma, including at least one long non-coding RNA (lncRNA) selected from the group consisting of RP11-114H23.1, HERES, RP11-1L12.3, CTD-2319I12.1, LINC00330 and RP11-114H23.2, and a use thereof.

Furthermore, an object of the present invention is to provide a composition for diagnosing or predicting the prognosis of squamous cell carcinoma and a kit for diagnosing or predicting the prognosis of squamous cell carcinoma, including a preparation capable of measuring an expression level of at least one long non-coding RNA (lncRNA) selected from the group consisting of RP11-114H23.1, HERES, RP11-1L12.3, CTD-2319I12.1, LINC00330 and RP11-114H23.2.

Further, another object of the present invention is to provide an information-providing method for diagnosing or predicting the prognosis of squamous cell carcinoma, the method including: measuring an expression level of a long non-coding RNA (lncRNA) selected from the group consisting of RP11-114H23.1, HERES, RP11-1L12.3, CTD-2319I12.1, LINC00330 and RP11-114H23.2 from a sample of a patient with squamous cell carcinoma and comparing the expression level of the long non-coding RNA with that of a control sample.

In addition, still another object of the present invention is to provide a method for diagnosing or predicting the prognosis of squamous cell carcinoma by a preparation including at least one long non-coding RNA (lncRNA) selected from the group consisting of RP11-114H23.1, HERES, RP11-1L12.3, CTD-2319I12.1, LINC00330 and RP11-114H23.2.

However, a technical problem to be solved by the present invention is not limited to the aforementioned problems, and other problems that are not mentioned may be clearly understood by a person skilled in the art from the following description.

### Technical Solution

To achieve the objects, the present invention provides a marker composition for diagnosing or predicting the prognosis of squamous cell carcinoma, including at least one long non-coding RNA (lncRNA) selected from the group consisting of RP11-114H23.1 (ENST00000552856.1, ENST00000552466.2), HERES, RP11-1L12.3 (ENST00000525302.1, ENST00000526061.1, ENST00000531363.1. ENST00000530430.1), CTD-2319112.1 (ENST00000566140.1, ENST00000590346.1, ENST00000587298.1, ENST00000592556.1, ENST00000590012.1, ENST00000589987.1, ENST00000588180.1, ENST00000589777.1), LINC00330 (ENST00000414562.1) and RP11-114H23.2 (ENST00000548702.1).

As an exemplary embodiment of the present invention, the HERES may include a base sequence represented by SEQ ID NO: 3 or SEQ ID NO: 4.

An exemplary embodiment of the present invention, the present invention provides a use of the composition.

Furthermore, the present invention provides a composition for diagnosing or predicting the prognosis of squamous cell carcinoma, including a preparation capable of measuring an expression level of at least one long non-coding RNA (lncRNA) selected from the group consisting of RP11-114H23.1 (ENST00000552856.1, ENST00000552466.2), HERES, RP11-1L12.3 (ENST00000525302.1, ENST00000526061.1, ENST00000531363.1, ENST00000530430.1), CTD-2319112.1 (ENST00000566140.1, ENST00000590346.1, ENST00000587298.1, ENST00000592556.1, ENST00000590012.1, ENST00000589987.1, ENST00000588180.1, ENST00000589777.1), LINC00330 (ENST00000414562.1) and RP11-114H23.2 (ENST00000548702.1).

As an exemplary embodiment of the present invention, the HERES may include a base sequence represented by SEQ ID NO: 3 or SEQ ID NO: 4.

Further, the present invention provides a kit for diagnosing or predicting the prognosis of squamous cell carcinoma, including the composition.

In addition, the present invention provides an information-providing method for diagnosing or predicting the prognosis of squamous cell carcinoma, the method including: a) measuring an expression level of at least one long non-coding RNA (lncRNA) selected from the group consisting of RP11-14H23.1 (ENST00000552856.1. ENST00000552466.2). HERES, RP11-1L12.3 (ENST00000525302.1, ENST00000526061.1, ENST00000531363.1, ENST00000530430.1). CTD-2319I12.1 (ENST00000566140.1, ENST00000590346.1, ENST00000587298.1, ENST00000592556.1, ENST00000590012.1. ENST00000589987.1, ENST00000588180.1, ENST00000589777.1), LINC00330 (ENST00000414562.1) and RP11-114H23.2 (ENST00000548702.1) from a sample of a patient with squamous cell carcinoma; and

b) comparing the expression level of the long non-coding RNA with that of a control sample.

As an exemplary embodiment of the present invention, the HERES may include a base sequence represented by SEQ ID NO: 3 or SEQ ID NO: 4.

As another exemplary embodiment of the present invention, when the expression level of HERES or RP11-1L12.3, which is the long non-coding RNA, is high, it is possible to predict that the risk of squamous cell carcinoma pathogenesis is high.

As still another exemplary embodiment of the present invention, when the expression level of at least one selected from the group consisting of RP11-114H23.1, CTD-2319I12.1, LINC00330 and RP11-114H23.2, which are the long non-coding RNA, is low, it is possible to predict that the risk of squamous cell carcinoma pathogenesis is high.

As yet another exemplary embodiment of the present invention, the expression level of the long non-coding RNA may be measured by at least one method selected from the group consisting of polymerase chain reaction (PCR), reverse transcription polymerase chain reaction (RTPCR), real-time polymerase chain reaction (Real-time PCR), RNase protection assay (RPA), a microarray, single molecule imaging, RNA FISH and northern blotting.

Furthermore, the present invention provides a method for diagnosing or predicting the prognosis of squamous cell carcinoma by a preparation capable of measuring an expression level of at least one long non-coding RNA (lncRNA) selected from the group consisting of RP11-114H23.1 (ENST00000552856.1, ENST00000552466.2), HERES, RP11-L12.3 (ENST00000525302.1, ENST00000526061.1. ENST00000531363.1. ENST00000530430.1), CTD-2319112.1 (ENST00000566140.1. ENST00000590346.1, ENST00000587298.1. ENST00000592556.1, ENST00000590012.1, ENST00000589987.1, ENST00000588180.1, ENST00000589777.1), LINC00330 (ENST00000414562.1) and RP11-114H23.2 (ENST00000548702.1).

As an exemplary embodiment of the present invention, the HERES may include a base sequence represented by SEQ ID NO: 3 or SEQ ID NO: 4.

### Advantageous Effects

The inventors discovered that the expression pattern of a long non-coding RNA is related to cancer pathogenesis, investigated the correlation between an expression of a particular long non-coding RNA and squamous cell carcinoma pathogenesis and thus confirmed that the long non-coding RNA can be a squamous cell carcinoma diagnostic or prognosis prediction marker. Therefore, the marker according to the present invention can assist management and treatment of a cancer patient by means of diagnosing or predicting the prognosis of squamous cell carcinoma.

### DESCRIPTION OF DRAWINGS

FIG. 1 shows a flowchart illustrating a process of discovering a marker for predicting the prognosis of esophageal squamous cell carcinoma.

FIG. 2 shows the results of classifying the long non-coding RNA expression pattern according to the present invention and patients with esophageal squamous cell carcinoma.

FIG. 3 shows the results of measuring the stage-free survival rates of patients with esophageal squamous cell carcinoma using HERES. RP11-1L12.3, CTD-2319112.1, RP11-114H23.1, RP11-114H23.2 or LINC00330 as a marker for esophageal squamous cell carcinoma in the patients with esophageal squamous cell carcinoma.

FIG. 4 shows the results of measuring the stage-free survival rates of patients with head and neck squamous cell carcinoma using HERES as a marker for head and neck squamous cell carcinoma in the patients with head and neck squamous cell carcinoma.

### MODES OF THE INVENTION

Hereinafter, the present invention will be described in detail.

The inventors discovered that the expression pattern of a long non-coding RNA is related to cancer pathogenesis, investigated the correlation between an expression of a particular long non-coding RNA and squamous cell carcinoma pathogenesis, and confirmed that the long non-coding RNA can be a squamous cell carcinoma diagnostic or prognosis prediction marker, thereby completing the present invention.

The present invention provides a marker composition for diagnosing or predicting the prognosis of squamous cell carcinoma, including at least one long non-coding RNA (lncRNA) selected from the group consisting of RP11-114H23.1, HERES, RP11-1L12.3, CTD-2319I12.1, LINC00330 and RP11-114H23.2; a composition for diagnosing or predicting the prognosis of squamous cell carcinoma, including a preparation capable of measuring an expression level of a long non-coding RNA; and a kit for diagnosing or predicting the prognosis of squamous cell carcinoma, including the composition.

The RP11-114H23.1 according to the present invention may include a base sequence represented by SEQ ID NO: 1 (ENST00000552856.1) or SEQ ID NO: 2 (ENST00000552466.2), the HERES may include a base sequence represented by SEQ ID NO: 3 or SEQ ID NO: 4, the RP11-1L12.3 may include any one base sequence selected from the group consisting of base sequences represented by SEQ ID NO: 5 (ENST00000525302.1), SEQ ID NO: 6 (ENST00000526061.1), SEQ ID NO: 7 (ENST00000531363.1) and SEQ ID NO: 8 (ENST00000530430.1), the CTD-2319I12.1 may include any one base sequence selected from the group consisting of base sequences represented by SEQ ID NO: 9 (ENST00000566140.1), SEQ ID NO: 10 (ENST00000590346.1), SEQ ID NO: 11 (ENST00000587298.1), SEQ ID NO: 12 (ENST00000592556.1), SEQ ID NO: 13 (ENST00000590012.1), SEQ ID NO: 14 (ENST00000589987.1), SEQ ID NO: 15 (ENST00000588180.1) and SEQ ID NO: 16 (ENST00000589777.1), the LINC00330 may include a base sequence represented by SEQ ID NO: 17 (ENST00000414562.1), and the RP11-114H23.2 may include a base sequence represented by SEQ ID NO: 18 (ENST00000548702.1). The characters shown in the parentheses indicate a transcript ID of a transcript described in GRCh37.p13.

The HERES according to the present invention may include a base sequence represented by SEQ ID NO: 3 or SEQ ID NO: 4, and in this case, may also include a base sequence having 70% or more, preferably 80% or more, more preferably 90% or more, and most preferably 95%, 96%, 97%, 98% or 99% or more sequence homology with the base sequence represented by SEQ ID NO: 3 or 4.

Here, an HERES gene encoding the HERES according to the present invention may include a base sequence represented by SEQ ID NO: 19.

“Squamous cell carcinoma”, which is the target disease of the present invention, is a malignant tumor derived from keratinocytes of the epidermis.

Squamous cell carcinoma is a non-melanoma skin cancer which is more complex than basal cell carcinoma in terms of biological aspects such as metastasis according to tumor size and depth, cause, anatomical location, and histological characteristics, and is one of the most commonly occurring skin cancers along with basal cell carcinoma in Korea. Further, squamous cell carcinoma occurs not only on the skin but also on the mucous membranes, its symptoms vary depending on the site of occurrence and the cause, and the cancer generally looks like broken flesh with a relatively large and uneven shape, and since the surface of the tumor (carcinoma) becomes weak at the onset of squamous cell carcinoma, infection by general bacteria is likely to occur, and although there may be pus or a foul odor, it is reported that there are no other subjective symptoms. Types thereof include squamous cell carcinoma arising from sun-damaged skin, and squamous cell carcinoma arising from cicatrices (scars) and chronic inflammatory diseases, arsenic-induced squamous cell carcinoma, radioactivity-induced squamous cell carcinoma, de novo squamous cell carcinoma or verrucous carcinoma depending on the cause, and include squamous cell carcinoma of the face, neck, arms, and the like, squamous cell carcinoma originating in the lungs, squamous cell carcinoma originating in the esophagus and squamous cell carcinoma originating in the head and neck depending on the site of occurrence, but are not limited thereto.

The “long non-coding RNA (lncRNA)” according to the present invention refers to a long non-coding RNA consisting of 200 nt or more among non-coding RNAs which are transcribed, but are not translated, on DNA sequences. It is known that the expression pattern thereof differs depending on the type of cell. According to recent studies, lncRNAs are emerging as a solution to precise transcriptional regulation according to the gene types that could not be revealed by epigenetics in the past while results that lncRNAs, as well as transcriptional regulators or enzymes such as enzymes for histone methylation, acetylation, phosphorylation and the like, help specific transcriptional regulation for each cell are being produced. Such lncRNAs have been reported to contribute to such transcriptional regulation through mechanisms such as inducing a specific protein to a specific DNA region by themselves or being attached to a specific protein to change the activity thereof. The “preparation” which measures the level of the long non-coding RNA according to the present invention is preferably an antisense oligonucleotide, a primer pair or a probe. The antisense refers to an oligomer having a nucleotide base sequence and a backbone between subunits, in which an antisense oligomer is hybridized with a target sequence in RNA by Watson-Crick base pairing to typically allow the formation of mRNA and RNA:oligomeric heteroduplexes within the target sequence. The oligomer may have exact sequence complementarity or approximate complementarity to the target sequence.

The “kit” according to the present invention may predict the pathogenesis or prognosis of squamous cell carcinoma by confirming the expression level of a long non-coding RNA, which is a marker whose expression is differentially increased in a group of patients with squamous cell carcinoma to confirm whether the long non-coding RNA is over- or under-expressed in a test subject, and by confirming the expression level of a long non-coding RNA whose expression is increased or decreased in a non-patient group to confirm whether the long non-coding RNA is over- or under-expressed in a test subject. That is, the kit may diagnose or predict the prognosis of squamous cell carcinoma by confirming the expression level of a long non-coding RNA and detecting it with a marker. A kit for detecting a marker according to the present invention may include not only a primer or probe for measuring the expression level of a long non-coding RNA which is a marker capable of predicting the presence or absence and prognosis of squamous cell carcinoma, but also at least one other component composition, solution and device suitable for analysis (a gene panel and the like), and the kit for measuring the expression level of a long non-coding RNA which is the marker may be a kit including essential elements required to perform RT-PCR, but is not limited thereto. In addition, the RT-PCR kit may include not only each primer pair specific for a long non-coding RNA which is a marker, but also a test tube or other appropriate containers, a reaction buffer, deoxynucleotides (dNTPs), enzymes such as Taq-polymerase and reverse transcriptase, DNase, a RNase suppressor, DEPC-water, sterilized water, and the like, but is not limited thereto.

In an embodiment of the present invention, six long non-coding RNAs that were significantly correlated with the pathogenesis of esophageal squamous cell carcinoma were selected (see Example 2), and it was confirmed that when HERES and RP11-1L12.3, which are long non-coding RNAs, were highly expressed in a group of patients with esophageal squamous cell carcinoma, the risk of esophageal squamous cell carcinoma pathogenesis was increased, and when CTD-2319I12.1, RP11-114H23.1, RP11-114H23.2 and LINC00330, which are long non-coding RNAs, were lowly expressed, the risk of esophageal squamous cell carcinoma pathogenesis was increased (see Example 3).

The above results confirm that HERES, RP11-1L12.3, CTD-2319I12.1, RP11-114H23.1. RP11-114H23.2 and LINC000330, which are long non-coding RNAs, can be used as esophageal squamous cell carcinoma diagnostic or prognosis prediction markers.

As another aspect of the present invention, the present invention provides an information-providing method for diagnosing or predicting the prognosis of squamous cell carcinoma, the method including a) measuring an expression level of a long non-coding RNA (lncRNA) selected from the group consisting of RP11-114H23.1. HERES, RP11-1L12.3, CTD-2319I12.1, LINC00330 and RP1-114H23.2 from a sample of a patient with squamous cell carcinoma and b) comparing the expression level of the long non-coding RNA with that of a control sample.

In the present invention, when the expression level of HERES or RP11-1L12.3 is high, it is possible to predict that the risk of squamous cell carcinoma pathogenesis is high.

In the present invention, when the expression level of at least one selected from the group consisting of RP11-114H23.1, CTD-2319I12.1, LINC00330 and RP11-114H23.2 is low, it is possible to predict that the risk of squamous cell carcinoma pathogenesis is high.

In the present invention, the expression level of the long non-coding RNA may be measured by at least one method selected from the group consisting of polymerase chain reaction (PCR), reverse transcription polymerase chain reaction (RTPCR), real-time polymerase chain reaction (Real-time PCR), RNase protection assay (RPA), a microarray, single molecule imaging, RNA FISH and northern blotting, but is not limited thereto.

The “information-providing method for diagnosing or predicting the prognosis of squamous cell carcinoma” according to the present invention refers to the provision of objective basic information required to diagnose or predict the prognosis of squamous cell carcinoma as a preliminary step for diagnosis or prediction of the prognosis, and a doctor's clinical determination or findings are excluded.

The “patient sample” according to the present invention includes blood and other liquid samples of biological origin, biopsy specimens, solid tissue samples such as tissue cultures, or cells derived therefrom. More specifically, the patient sample may be a tissue, an extract, a cytolysate, whole blood, serum, and the like, but is not limited thereto.

Hereinafter, preferred Examples for helping the understanding of the present invention will be suggested. However, the following Examples are provided for easier understanding of the present invention, and the contents of the present invention are not limited by the following Examples.

### EXAMPLES

### Example 1. Experimental Preparation and Experimental Methods

After esophageal tissue excised from a patient with esophageal squamous cell carcinoma was subjected to homogenization. RNA was extracted to perform RNA analysis. For RNA extraction, cell constituents excluding RNA, DNA and proteins were removed using Trizol reagent, and then RNA, DNA and proteins were isolated using chloroform. After an RNA layer was extracted from the separated layer, RNA was washed and deposited. After pellets were made by precipitating RNA, an RNA sample was secured by dissolving the pellets in nuclease-free water. Sequencing was performed from the RNA sample using Illumina Hiseq 2000.

### Example 2. Selection of Squamous Cell Cancer Diagnostic or Prognosis Prediction Marker Long Non-Coding RNA

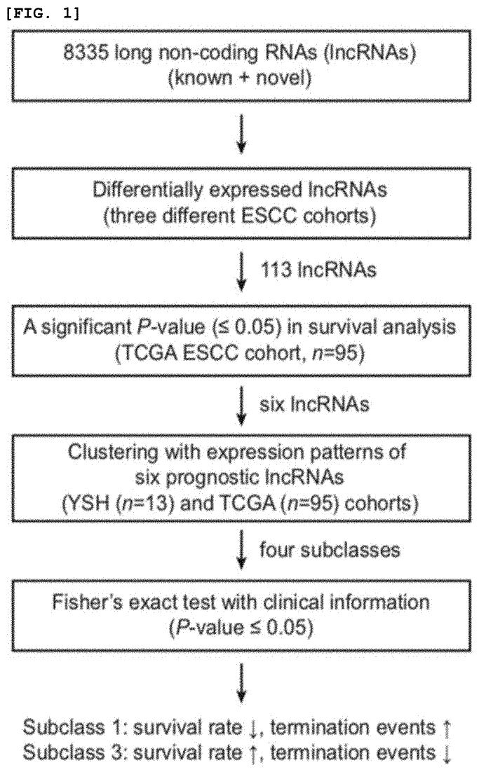

A long non-coding RNA, which is a marker to be used for diagnosing or predicting the prognosis of esophageal squamous cell carcinoma, was selected by the following process as illustrated in FIG. 1.

2-1. Confirmation of Changes in Expression Pattern of Long Non-Coding RNA in Korean Esophageal Squamous Cell Carcinoma RNA Sequencing Data

An expression pattern analysis was performed on 8335 long non-coding RNAs using the most commonly used annotation data GENCODE (version 19) and a long non-coding RNA (lncRNA) newly identified in RNA sequencing data of Korean patients with esophageal squamous cell carcinoma.

2-2. Selection of Long Non-Coding RNA with Commonly Changing Expression Pattern from Independent Korean, Chinese and TCGA Patient Groups

Based on the results in Example 2-1, long non-coding RNAs with a commonly changing expression pattern in Korean, Chinese and TCGA patient groups were selected in order to find long non-coding RNAs specifically over- or under-expressed in patients with esophageal squamous cell carcinoma.

As a result, as illustrated in FIG. 1, 113 long non-coding RNAs were selected.

2-3. Selection of Squamous Cell Cancer Diagnostic or Prognosis Prediction Marker Long Non-Coding RNA from Group of Patients with Esophageal Squamous Cell Carcinoma of TCGA

Based on the results in Example 2-2, each long non-coding RNA expression pattern and 5 year survival rates of patients were analyzed in a group (n=95) of patients with esophageal squamous cell carcinoma of TCGA including various patient backgrounds.

As a result, as illustrated in FIG. 1, it was confirmed that a total of six long non-coding RNAs (HERES, RP11-1L12.3, CTD-2319I12.1, RP11-114H23.1, RP11-114H23.2, LINC00330) showed a significant correlation with the prognosis of patients.

From the above results, it could be confirmed that HERES, RP11-1L12.3, CTD-2319I12.1, RP11-14H23.1, RP11-114H23.2 and LINC00330, which are long non-coding RNAs, could be used as esophageal squamous cell carcinoma diagnostic or prognosis prediction markers.

### Example 3. Application of Squamous Cell Cancer Diagnostic or Prognosis Prediction Marker

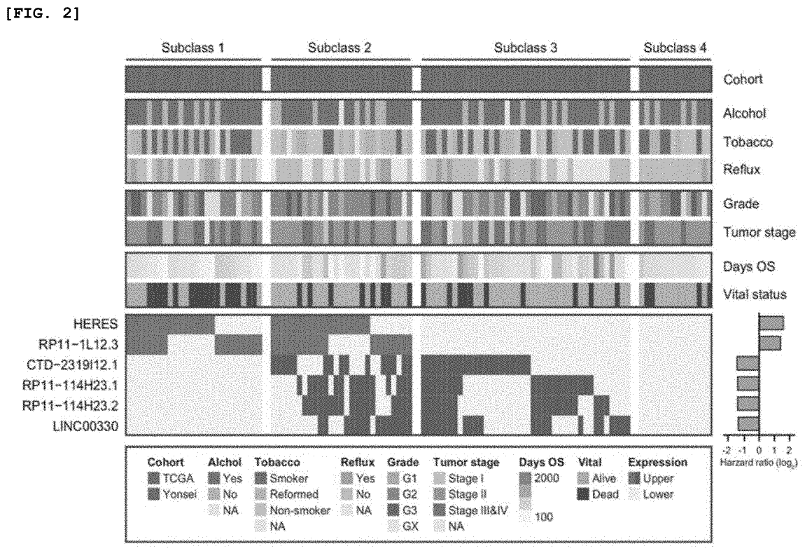

Based on the results in Example 2, as illustrated in FIG. 2, the groups of patients with esophageal squamous cell carcinoma of Koreans (n=13) and TCGA (n=95) were classified into four groups based on the expression pattern of the six long non-coding RNAs (subclass 1, 2, 3 and 4).

Further, a correlation between the patient survival rate of the four groups and the expression level of the above six long non-coding RNAs was confirmed.

As a result, as illustrated in FIG. 2, it was confirmed that the survival rate of a patient group (subclass 1) with high expression of HERES and RP11-1L12.3 and low expression of CTD-2319I12.1, RP11-114H23.1, RP11-114H23.2 and LINC00330 was low, but the survival rate of a patient group (subclass 3) with low expression of HERES and RP11-1L12.3 and high expression of CTD-2319I12.1, RP11-14H23.1. RP11-114H23.2 and LINC00330 was high.

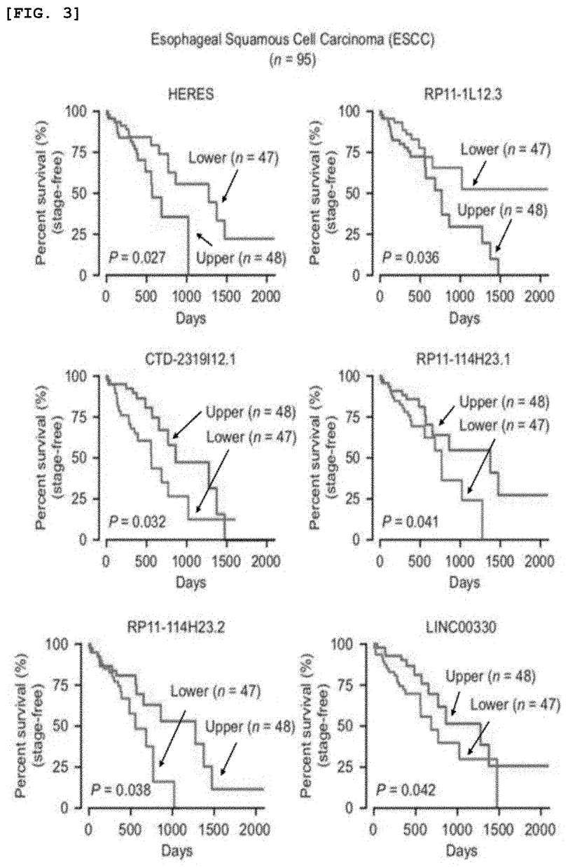

In addition, as illustrated in FIG. 3, stage-free survival rates of patients with esophageal squamous cell carcinoma for 5 years were measured using HERES, RP11-1L12.3, CTD-2319I12.1, RP11-114H23.1, RP11-114H23.2 or LINC00330 as a marker in the patients with esophageal squamous cell carcinoma.

As a result, it was confirmed that the lower the expression rate of HERES or RP11-1L12.3 was, the higher the survival rate of patients was, and the higher the expression rate of CTD-2319I12.1, RP11-114H23.1, RP11-114H23.2 or LINC00330 was, the higher the survival rate of patients was.

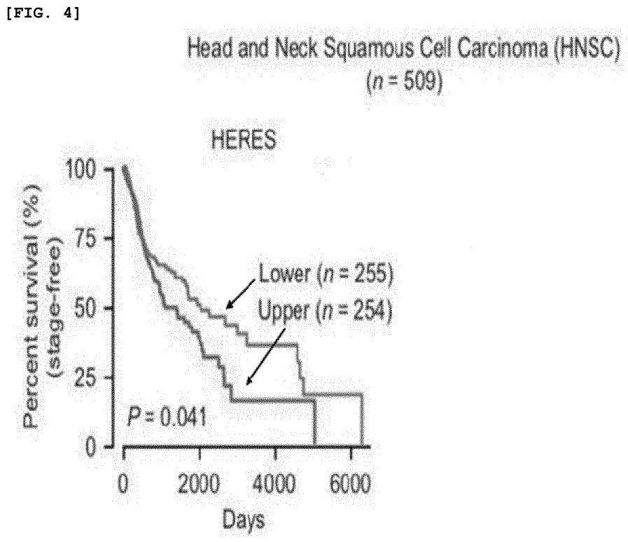

Furthermore, as illustrated in FIG. 4, stage-free survival rates of patients with head and neck squamous cell carcinoma for 5 years were measured using HERES as a marker in the patients with head and neck squamous cell carcinoma.

As a result, it was confirmed that the lower the expression rate of HERES was, the higher the survival rate of patients was.

From the above results, it could be confirmed that the high expression of HERES or RP11-1L12.3, which is a long non-coding RNA, is related to a high risk of esophageal squamous cell carcinoma pathogenesis, and the low expression of CTD-2319I12.1, RP11-114H23.1. RP11-114H23.2 or LINC00330, which is a long non-coding RNA, is related to a high risk of esophageal squamous cell carcinoma pathogenesis.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only illustrative in all aspects and not restrictive.

### INDUSTRIAL APPLICABILITY

The present invention is a technology capable of developing a long non-coding RNA which is related to squamous cell carcinoma as a composition capable of diagnosing or observing the prognosis of squamous cell carcinoma using the long non-coding RNA, and since it is possible to more rapidly and precisely diagnose or observe the prognosis of the carcinoma by a non-invasive test compared to diagnostic markers in the related art, the technology according to the present invention can be widely used in the development fields such as the management and diagnosis of the carcinoma.

1. A method for diagnosing or predicting the prognosis of squamous cell carcinoma by using a preparation capable of measuring an expression level of at least one long non-coding RNA (lncRNA) selected from the group consisting of RP11-1141H23.1 (ENST00000552856.1, ENST00000552466.2), HERES, RP11-1l12.3 (ENST00000525302.1, ENST00000526061.1, ENST00000531363.1, ENST00000530430.1), CTD-2319I12.1 (ENST00000566140.1, ENST00000590346.1, ENST00000587298.1, ENST00000592556.1, ENST00000590012.1, ENST00000589987.1, ENST00000588180.1, ENST00000589777.1), LINC00330 (ENST00000414562.1) and RP11-1141H23.2 (ENST00000548702.1).

2. The method of claim 1, wherein the HERES comprises a base sequence represented by SEQ ID NO: 3 or SEQ ID NO: 4.

3. (canceled)

4. (canceled)

5. A kit for diagnosing or predicting the prognosis of squamous cell carcinoma, comprising a preparation capable of measuring an expression level of at least one long non-coding RNA (lncRNA) selected from the group consisting of RP11-114H23.1 (ENST00000552856.1, ENST00000552466.2), HERES, RP11-1L12.3 (ENST00000525302.1, ENST00000526061.1, ENST00000531363.1, ENST00000530430.1), CTD-2319I12.1 (ENST00000566140.1, ENST00000590346.1, ENST00000587298.1, ENST00000592556.1, ENST00000590012.1, ENST00000589987.1, ENST00000588180.1, ENST00000589777.1), LINC00330 (ENST00000414562.1) and RP11-114H23.2 (ENST00000548702.1).

6. An information-providing method for diagnosing or predicting the prognosis of squamous cell carcinoma, the method comprising: a) measuring an expression level of a long non-coding RNA (lncRNA) selected from the group consisting of RP11-114H23.1 (ENST00000552856.1, ENST00000552466.2), HERES, RP11-1L12.3 (ENST00000525302.1, ENST00000526061.1, ENST00000531363.1, ENST00000530430.1), CTD-2319I12.1 (ENST00000566140.1, ENST00000590346.1, ENST00000587298.1, ENST00000592556.1, ENST00000590012.1, ENST00000589987.1, ENST00000588180.1, ENST00000589777.1), LINC00330 (ENST00000414562.1) and RP11-114H23.2 (ENST00000548702.1) from a sample of a patient with squamous cell carcinoma; and b) comparing the expression level of the long non-coding RNA with that of a control sample.

7. The information-providing method of claim 6, wherein the HERES comprises a base sequence represented by SEQ ID NO: 3 or SEQ ID NO: 4.

8. The information-providing method of claim 6, wherein when an expression level of HERES or RP11-1L12.3, which is the long non-coding RNA, is high, it is predicted that a risk of squamous cell carcinoma pathogenesis is high.

9. The information-providing method of claim 6, wherein when an expression level of at least one selected from the group consisting of RP11-114H23.1, CTD-2319I12.1, LINC00330 and RP11-114H23.2, which are long non-coding RNAs, is low, it is predicted that a risk of squamous cell carcinoma pathogenesis is high.

10. The information-providing method of claim 6, wherein an expression level of the long non-coding RNA is measured by at least one method selected from the group consisting of polymerase chain reaction (PCR), reverse transcription polymerase chain reaction (RTPCR), real-time polymerase chain reaction (Real-time PCR), RNase protection assay (RPA), a microarray, single molecule imaging, RNA FISH and northern blotting.

11. (canceled)

12. (canceled)

13. (canceled)

14. (canceled)


SEQUENCE LISTING (19 sequences)

SEQ ID NO: 1 - DNA, 748 bp, Homo sapiens
  Encodes the transcipt RP11-114H23.1-001 ENST00000552856.1
gtggaagtgaggaagagggagagctcctgggcaccagaatgaatggggagggtcctttagggcactgtgaggccattagcttttactcctactgagactggacatcccaggagagtttcagagcagagaagtgctatgactgctcttgaagtttaagaggaccaggtggctggctttgctgtgtgtcagagaacaggagcagaaacagggagaccagctgggaggtgactgcaaaagtccaggtgagaggcgatggagctagtgaggagtgccggaggcctggtgtattctgacggtgccaggacctgtgataggtgctgagagagatgaggaactgctcagatcaccagctgcttcaggttaccagcccacacctgtctgtctcacctctgacaaccttggtaaagaaaccaaggcttagagacctgctcacagtcgtttcgtgagggaatgcaacatctgggtctagggtccaaatctatctctgctgtgaagaggaactcatacaggagatctacatgattaccaattcttggccttgcagtggaggatattaattttaagtacctgtgaaatgaagttcttaaaacctaagattccccaaaactccagttatcaacaaggttatataggaatcaaagcgaggcgtttggagtagatgccgagatcacctctccctcacacctctgctgaccccagctacctgcaatcctgccaggaccattctggaggtgaggtgatccttttt

SEQ ID NO: 2 - DNA, 712 bp, Homo sapiens
  Encodes the transcipt RP11-114H23.1-003 ENST00000552466.2
actacttctccagattctgaatgttctacatctgggcacaagattaccgttctttcattgtaccatctcaggctcagtcattgttacttaagcaggccaccatgttgcgggactggtggggatgagcttgcaggctcagttgtcagtgtgagaactttccctttcctatgaccttccagatgaggaactgctcagatcaccagctgcttcaggttaccagcccacacctgtctgtctcacctctgacaaccttgatataaattaaacaatgaataagaacaaaaacaatttaaaactgcgtaatggtccaaaccaccatggatggtgttctgacgatgatacacggggtgaggtctgactcatgagccgctgtccgtgatgacttgacattctcaaaaggaaatcccgaatgttctcactgcatgctgctgcaggccctcatggcagggcatccacaccactggctcaggtgctggttgctccagaatctctcttcagctggcctttttctactgaactgtctgtgagaacatcactgcaggcacggttttctccaaccataattgctgctgccaaagccacctcggacttgggaaagggaaacaacactccactagagagcgtagaatcttagaattcatctacttcattctcctcctggtactctactttcttctacaacgtcccagccgagctgaggtctgagctgtgc

SEQ ID NO: 3 - DNA, 2160 bp, Homo sapiens
  HERES.1 gene
ggcaaccagcttgcgtccgtttccatgttgtgggagatttgttctttcgctctttgtggtaaatcttgctactgctcactctttgagtccacactgcttttatgagctgtaacactcaccgggaaggtctgcagcttcaatcctgaagccagcgaggccaccagcccaccgggaggaacgaacaactccagacgcaccgccttaagagctgtaacactcaccgcgaaggtctgcagcttcactcctgagccagcgagaccacgaacccaccagaagaaagaaactccgaacacatccgaacaccagaaggaacaaactccagacgcgccaactgtaacactcaccgcgaggatccgtggcttcattcttgaagtcagtgagaccaagaacccaccaattccggacacactgtgagtcaggatcagtcttattatagctaattagagcaaccccattgctaaagagtttagagcctgggtcagacttcatggtagcaaagcaatgttttattgtaatagtgtaattaggaatgtgtggcaatattttaacccctcctcccaagagttccacaatgctttcttaacatgtttataaatatttgcttttgtgccattttaggggagctttccgtacaaacatttggcttttaatgagtcatccacactctgattcatacccataagcttcctgctttgttatcagtgaatattgggctcagtaactgccttacttttataatttacatgaatgaatatggaaaaggtggtggaggtagaaagacatattcaggtaaatataaaaacaccctggtttctttacaagagattgacaaatacggcccaataccctatcatttaaaaaaatagctgctgccccactcctgtgccaaaatctgctccaagggatcctatcctagtcagaaggctgtactctgaatgctgatattagctacaacaccaaggcttcccactatacaaactggatcctcaaagtcatccacaccaaatgaactctctattctttctcccagtagcctgagaactgggtttaccagccttggcccttcctttccttctagcctcaaaacaggatgggaacagagtctttggctgtgagaatgggctcaataacaaaataaagatgacagaagagtcagtgacttcaatgacagagcgatagaaattaccaatataaacggagagaaaagagattttttaaaaatgaacaaatccccaaggacctgtgagacaataactgtgttaggttttctaaacttacaagtgtttttatttatatatggtaagcaactatgcattatttgtgtcagaattattttttgtatatatttaaggtatacaacatgatgtctcaacatacttatatgtagtgaaatggttactacagtcaccacacttattgtctcacaggttattgtcttcagaaggagagtaaaaagaatgctgggtagaagaaatacctgaagaaataatggcccaacctcaaatttggtgaaagacataaacttacagattcaagaaactgagtgttccccaaacagaataaactttaaagaatccacactcaaacacatcataatcaaacttctgaaaactaacaacaaagaaaaacttaaaatcagcaagagagaaaccacacattatcaactgatgaaaagtgactcaagtgacagcagatttataatcagaaacttgaaggacagaaggaagaggcaaaacattttgaaagccctaaaaggaaagaactgtcaacccagaattcgacatccagtaaaaatatccgtcaagaatgaatatgaaggaaagctaagaatgaatatatcttctatttctttgctaaatttttctactttttcatttgatgtaagagtgtttataagtgcttatggagcatttttatgatagttggtttacaatccttgtctgagagtagctatgtgagatgacgtatatagtaaatggtgactgtagtaaccatttcactacatataagtatgtcaaaacatgttgtacaccttaaatatacacaaaaaatagctctgacacaaaaaatgcatagttgttattaccatgtataaataaaaatacttgtaagtttagaaaataaaatcattgtctgataattatctaaataataaagtcc

SEQ ID NO: 4 - DNA, 6675 bp, Homo sapiens
  HERES.2 gene
ggcaaccagcttgcgtccgtttccatgttgtgggagatttgttctttcgctctttgtggtaaatcttgctactgctcactctttgagtccacactgcttttatgagctgtaacactcaccgggaaggtctgcagcttcaatcctgaagccagcgaggccaccagcccaccgggaggaacgaacaactccagacgcaccgccttaagagctgtaacactcaccgcgaaggtctgcagcttcactcctgagccagcgagaccacgaacccaccagaagaaagaaactccgaacacatccgaacaccagaaggaacaaactccagacgcgccaactgtaacactcaccgcgaggatccgtggcttcattcttgaagtcagtgagaccaagaacccaccaattccggacacactgtgagtcaggatcagtcttattatagctaattagagcaaccccattgctaaagagtttagagcctgggtcagacttcatggtagcaaagcaatgttttattgtaatagtgtaattaggaatgtgtggcaatattttaacccctcctcccaagagttccacaatgctttcttaacatgtttataaatatttgcttttgtgccattttaggggagctttccgtacaaacatttggcttttaatgagtcatccacactctgattcatacccataagcttcctgctttgttatcagtgaatattgggctcagtaactgccttacttttataatttacatgaatgaatatggaaaaggtggtggaggtagaaagacatattcaggtaaatataaaaacaccctggtttctttacaagagattgacaaatacggcccaataccctatcatttaaagtaagtgccagaattgtgatggatttgtgtttggccttaaaaaaaaataaaaataaaaataaaaacaaaaacacttggataattctacctgatgttacatttttctttttttttttcttttctttttttttttttttttttgagatggagttttcctctgtcacccaggctggagtgcagtgacgtgacctcagctcgctgcaacctctgcctcccagattcaagcaattctctctcaaattgctcagaagctgagcaattctgcctcagcttcccaaatagctgggattacaggtgcgtgccaccaggctcagctaatttttgtatttttagtagagatggggtttcaccatgttggccaggctggtcttgaactcccgacctcaggtgatacgcccgcctcggcctcccaaagtgctgggattacaggcatgagccactgcgcccagcctgatgttccatttttctaatcaccaagtgtatttattagctaagggtatcccagaaattaaattaccaacataaaattttacttgtgctattataatttagctattaaatcctaaaattatcaactgaaagctactggaaaaataaatacttctctttcttttctccatattgaaagtaaagagaatgaggatccttagctaagattttaattaaatcttttctgagccagaattgcaaagcttttctttgtataaataaggactcgagttataaagcatagaactagcaatgagtctatcttggtccatttggattgctataaaggagtacctgaggctgggtaatttataaagaaaagacgtttattcgacacatgattctgcaggatgtacaagaagcatggcaccagcatctgctcttggtgaaaacttcaggtggctttgactcctggtggaagaagggaagtctagcttgtgcagagctcccatggccagagaaggaaggggggtttgaggaggtgccaggctctttgaatagagcgagaactcactcattaccaagagaaggcaccaagccattcatgagggatacacccctgtgacccaaaccttgccccccttccaacattggggcttaaatttcaatgtgatgtttaggggtacaaacatccaaaccacagcagagaaatacagagttctgtatttctattcccaaagggcaaaatttgggaggtgaatgggtctatatgtgtgttggggggaagaagttaatatgaagaaaagaaactaaagtgctttacataataatgagaattacagattaaaaaatctgtaatctttccctaaaaagggaaagaagtaccatgcaaattatgaggtaagtagactatatgacttatttatttttattaggttagagcaagaattgcaaactcaaggtcctgactccttgaattgcactctatggacccagtcatacttcatttgcatattatatattaaatgctttggcatgatctttgctcccccttacatcctcaaaacactctttttccatttttcttaaaatatatgttcctctttgtctatattttatttttccaactttgaaaaggacttacacataaaaatggtatcactttcttcttaactgtaagaaaaatggtctcagccatacattgatgaatggcagctgtcattcagcctcagtgtgaaggagacaacaatgagtggtgaggactgtaataaaatggaaagcacatgcccatccaaaagtgagcattcacttctaggaactagtaaaaatggtaatagccagatctttctattttttcacaagaagcctgaaagtatttaaatattttatgactaattcaaatgctttaaaaataagcaatttgattctaggaggccaaactagttgctatctatgatttaaaccatagtgtctttcagaattaatgtatgtccttggcgggtgtgtgtgtgtgtgtgtgtgtgtgatcccaaaacaccaataattaccagtgaaaaatttaggagattaaatatgtgtactttaatccacattgggggaagagagagatataaaaggtgctgagaaccttgaagtggtgcccctcctgatctgccttctacctcttcaacatacgtgctgtgcaccatgaaagacttgaagtgcaatggcaatctgtgatcctttgtggaaacctttccctaatttgccccagagtgaatgtttcacacttggattcacatttgctgagagaggcggaaaaatcttagggacaattagaacaataataacaacattcatattattattcaatgacagcaacattcatattcattctttcattcagcaaatatttattgcacacttgctatgtgctagaactataaaatggcaatgatgtgttaggtaatttgtaatcatggggaaaaaatcagttagggaaagacaaaactgccttcccttatagagattatattcattgaggagacaaataataaataaaatgaatagtgtaacataggttagatagtgacaagtgctatggagaaaaagaaagtaggaaagggtcatttctctggtttcctgatgtgtaattggaatagatatccttggtagctggtagaatctctgcagtgattccttgtactgtggggtaaaagctatgttagcaggggagaccaagaggaatagctgaaacaaaattcactttctcagtaagatagtaaataagaaagtgatattgcatcctgggaaacatggccgagattagtgcttctctttaagacctaaaggatgcagagtgatggccctcactcatcctcatttaattcacctgtcaggcccttgcaaaaattggtctgatcctgggggataataaactcaaccaaatagttgccccaattgcttttgttatgcgagatatatctttgctagaacaaactgattaacctggcctcaggtaccttgcattgattggcaaatgtattcatttctattcctgtaaagaaagagaaacaaaagcacgggaatgacacagacttttacagcactgagcagggctaattctcatgccctctgtcataatataacctaaagggacttggactgtctggatactccgcacatcaacaccataacccactatactgatgatatcatgtgtgatggttttcaaatatgcacacaaatttctgattcttctttcttcaagaggtggagcttaattccccttcaattgagggtgagtcgggcatagtgacttgcttctaattaataaaatattgcagaagtgatgatgtggtttttctaagacactgcaacttcctttccttgctctctcttgaataacatgctctgaaggaaatcaggtactatgtgatgaggacattcaagcaatcctagggagagattcatgtagaaaagaactaaggcctcctgccaaaagccatttgagtcagccctcttggaagtaaaccctctacccctagtaaagccttcggatgactgcagccctgggcagcatcttgactgcaaactcatgagagaccctgagccacaatcacccagctaagccactcttaaattcctgtcctatactgtgagataataaatgtttactggtttcagctgaaaggtgacagagtgggagtcatgtcaccatctctggctgtacacggaagaattacagtgagtgacctttaacacaaccccttagtgctccttcggtacccagtaaatgagggttatcatacaactatgattatagagcaatagatctggcttattcatgagacacttctgagcagtcataaattaaagcatgcatgcaaaagcccaagatatgccgttaacaacactggaaccagacctcggttttctcactttattatctgttctcctcttctttggtttctttttattttgtttttttccatcattgaatgatggtgaagttaataatggaaaagagggagggaagaagagaagaaaggaagttgaatatatgaaggaccaaagagaatattaccttcctccttctcgggtgtgcttagacacaaaacctaacatccaaataaaccaaggtgtcatttggatctctaatggagattgccaagagttctgtatgagacagattcctattaaagtctcagaacacgttctgtgcaagatcatctctggagagcatatatgttgggaaacccttttccctctgccttccctaaatttggtctggaacactgttttggggtccacacatatccttcaacccaacacaatctcaacacaagaagagatcagggtaaatggggatcaggaaggaatatgtatccttcttccagtcatgaatgaacttagatatcagggggacaaatttctgagtgttatttctccatggcttcccctgatgtggcctaagtgggtgaggaggcatttttcccagcccttatcacccaagtctttctattctggcccctcagatagagacttggtaatacatcacttttgttttggctgccctgcctgacccacctctcctctttctaacagaaaatagctgctgccccactcctgtgccaaaatctgctccaagggatcctatcctagtcagaaggctgtactctgaatgctgatattagctacaacaccaaggcttcccactatacaaactggatcctcaaagtcatccacaccaaatgaactctctattctttctcccagtagcctgagaactgggtttaccagccttggcccttcctttccttctagcctcaaaacaggatgggaacagagtctttggctgtgagaatgggctcaataacaaaataaagatgacagaagagtcagtgacttcaatgacagagcgatagaaattaccaatataaacggagagaaaagagattttttaaaaatgaacaaatccccaaggacctgtgagacaataactgtgttaggttttctaaacttacaagtgtttttatttatatatggtaagcaactatgcattatttgtgtcagaattattttttgtatatatttaaggtatacaacatgatgtctcaacatacttatatgtagtgaaatggttactacagtcaccacacttattgtctcacaggttattgtcttcagaaggagagtaaaaagaatgctgggtagaagaaatacctgaagaaataatggcccaacctcaaatttggtgaaagacataaacttacagattcaagaaactgagtgttccccaaacagaataaactttaaagaatccacactcaaacacatcataatcaaacttctgaaaactaacaacaaagaaaaacttaaaatcagcaagagagaaaccacacattatcaactgatgaaaagtgactcaagtgacagcagatttataatcagaaacttgaaggacagaaggaagaggcaaaacattttgaaagccctaaaaggaaagaactgtcaacccagaattcgacatccagtaaaaatatccgtcaagaatgaatatgaaggaaagctaagaatgaatatatcttctatttctttgctaaatttttctactttttcatttgatgtaagagtgtttataagtgcttatggagcatttttatgatagttggtttacaatccttgtctgagagtagctatgtgagatgacgtatatagtaaatggtgactgtagtaaccatttcactacatataagtatgtcaaaacatgttgtacaccttaaatatacacaaaaaatagctctgacacaaaaaatgcatagttgttattaccatgtataaataaaaatacttgtaagtttagaaaataaaatcattgtctgataattatctaaataataaagtcc

SEQ ID NO: 5 - DNA, 583 bp, Homo sapiens
  Encodes the transcipt RP11-1L12.3-001 ENST00000525302.1
agtttgatctcagactgctgtgctagcaatcagcgagactccgtgggcgtaggaccctccgagccagatgaagatcctgaataccaaagagggccgctgacaggtctaggagtacacttctagcacctagcagagagaggcttcactacatcatgcttcctgacatctctccctttgaagagcagtcagactcctgctttgctcttcagacttaatttgggggtttaacagccatgtgaagtgctcactccccctttgccttctgccatgattgtgtgtttcctgaggcctccccagaagctgagaagatgcttcctgtacagcctgcagaactggatagcccagagctattccatgtattccatggatgggcacatttggaagtttggttctaaaatctaaaagaagaaatttaagtgtgctgaagataactgcaactccaaacctaacgctgtgtaattctatgttctgccattttgaagcaaagtggaaccagaagtgagaggactatttctaggtcatgctacggttaccctgaccccagtcactccctatgctggtccatccaaactcagagccacga

SEQ ID NO: 6 - DNA, 471 bp, Homo sapiens
  Encodes the transcipt RP11-1L12.3-002 ENST00000526061.1
aaactctgcctgcttttagatggctcctcttagaactatgcatctctcctgatacttggatgcttttcctctgactgatgaagatcctgaataccaaagagggccgctgacaggtctaggagtacacttctagcacctagcagagagaggcttcactacatcatgcttcctgacatctctccctttgaagagcagtcagactcctgctttgctcttcagacttaatttgggggtttaacagccatgtgaagtgctcactccccctttgccttctgccatgattgtgtgtttcctgaggcctccccagaagctgagaagatgcttcctgtacagcctgcagaactgtttgaatgagagaggcagaagcacatttccaatcactgggggaacctgagaccattctttccaacccattttgccaaatggatgtcctgaggaaggtgttccaagagaggcgatgtcaggatagtg

SEQ ID NO: 7 - DNA, 745 bp, Homo sapiens
  Encodes the transcipt RP11-1L12.3-003 ENST00000531363.1
agactgctgtgctagcaatcagcgagactccgtgggcgtaggaccctccgagccaggtgcgggatataatctcgtggtgcgacgctttttaagcccttcagaaaagcgcagtgttcgggtgggagtgacccgattttccaggtgccgcccgtcacccctttctttgactcagaaagggaactccctgaccccttgcgcttcccaagtgaggcaatgcctcgccctgcttcggctcgcgcacggtgcgcgcacccgctgacctgcacccactgtctggcactccttagtgagatgaacccggtacctcagatggaaatgcagaaatcacccgtcttctgcgtcgctcacgctgggagctgtagaccggagctgttcctattcggccatcttggctcctcctgtaacaatttcttgagatgaagatcctgaataccaaagagggccgctgacaggtctaggagtacacttctagcacctagcagagagaggcttcactacatcatgcttcctgacatctctccctttgaagagcagtcagactcctgctttgctcttcagacttaatttgggggtttaacaggtgaggttgctgggggaactcttttacaacatctctctgaaagaatccgggctgccagtttcatttggtttgggtgtcagtagcatgatggaaagacaaaaaaacacaacttgacatctgcagaaatgggttcaaattttacctgcaactcaccaattctgtggc

SEQ ID NO: 8 - DNA, 879 bp, Homo sapiens
  Encodes the transcipt RP11-1L12.3-004 ENST00000530430.1
aatcagcgagactccgtgggcgtaggaccctccgagccaggtgcgggatataatctcgtggtgcgacgctttttaagcccttcagaaaagcgcagtgttcgggtgggagtgacccgattttccaggtgccgcccgtcacccctttctttgactcagaaagggaactccctgaccccttgcgcttcccaagtgaggcaatgcctcgccctgcttcggctcgcgcacggtgcgcgcacccgctgacctgcacccactgtctggcactccttagtgagatgaacccggtacctcagatggaaatgcagaaatcacccgtcttctgcgtcgctcacgctgggagctgtagaccggagctgttcctattcggccatcttggctcctcctgtaacaatttcttgagatgaagatcctgaataccaaagagggccgctgacaggtctaggagtacacttctagcacctagcagagagaggcttcactacatcatgcttcctgacatctctccctttgaagagcagtcagactcctgctttgctcttcagacttaatttgggggtttaacagccatgtgaagtgctcactccccctttgccttctgccatgattgtgtgtttcctgaggcctccccagaagctgagaagatgcttcctgtacagcctgcagaactggatagcccagagctattccatgtattccatggatgggcacatttggaagtttggttctaaaatctaaaagaagaaatttaagtgtgctgaagataactgcaactccaaacctaacgctgtgtaattctatgttctgccattttgaagcaaagtggaaccagaagtgagaggactatttctaggtcatgctacggttaccctgaccccagtc

SEQ ID NO: 9 - DNA, 447 bp, Homo sapiens
  Encodes the transcipt CTD-2319I12.1-001 ENST00000566140.1
cagcaacatgaagttggcagccttcctcctcctgtgatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagca

SEQ ID NO: 10 - DNA, 630 bp, Homo sapiens
  Encodes the transcipt CTD-2319I12.1-002 ENST00000590346.1
gggggaagaagaaaagaggatgccagtccttgttcctaggccaagacctgagccctgtaatattaatatttggatctaaaatcagccttccaacagcaacatgaagttggcagccttcctcctcctgtgatcctcatcatcttcagcctagaggtacaagagcttcaggctgcaggagaccggcttttgggtgtctgacccttctcaacacagcatcagacagccacctctattgactgaagttgacaatggagaataaaactcattacccctgctgaatccaggggcccctttttacagatgagaaaagttccagaaagacccagagacataatcaggtacctgcgtcgagctctgcacaggtgactgggactgcaaccccggagaccactgtgtcagcaatgggtgtggccatgagtgtgttgcaggatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagca

SEQ ID NO: 11 - DNA, 630 bp, Homo sapiens; identical to SEQ ID NO: 10
  Encodes the transcipt CTD-2319I12.1-003 ENST00000587298.1

SEQ ID NO: 12 - DNA, 474 bp, Homo sapiens
  Encodes the transcipt CTD-2319I12.1-004 ENST00000592556.1
gtgtttttccaaagccttccaacagcaacatgaagttggcagccttcctcctcctgtgatcctcatcatcttcagcctagaggtacaagagcttcaggctgcaggagaccggcttttgggggcccctttttacagatgagaaaagttccagaaagacccagagacataatcaggtacctgcgtcgagctctgcacaggtgactgggactgcaaccccggagaccactgtgtcagcaatgggtgtggccatgagtgtgttgcagggtaaggacagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagc

SEQ ID NO: 13 - DNA, 561 bp, Homo sapiens
  Encodes the transcipt CTD-2319I12.1-005 ENST00000590012.1
gtttttccaaagccttccaacagcaacatgaagttggcagccttcctcctcctgtgatcctcatcatcttcagcctagaggtacaagagcttcaggctgcaggagaccggcttttgggtgtctgacccttctcaacacagcatcagacagccacctctattgactgaagttgacaatggagaataaaactcattacccctgctgaatccaggggcccctttttacagatgagaaaagttccagaaagacccagagacataatcaggtacctgcgtcgagctctgcacaggtgactgggactgcaaccccggagaccactgtgtcagcaatgggtgtggccatgagtgtgttgcagggtaaggacagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgtta

SEQ ID NO: 14 - DNA, 558 bp, Homo sapiens
  Encodes the transcipt CTD-2319I12.1-006 ENST00000589987.1
atcctcatcatcttcagcctagaggtacaagagcttcaggctgcaggagaccggcttttggatactgctaatcagtcctagcactctttctctttgaacctggacaaggtgtctgacccttctcaacacagcatcagacagccacctctattgactgaagttgacaatggagaataaaactcattacccctgctgaatccaggggcccctttttacagatgagaaaagttccagaaagacccagagacataatcaggtacctgcgtcgagctctgcacaggtgactgggactgcaaccccggagaccactgtgtcagcaatgggtgtggccatgagtgtgttgcagggtaaggacagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagca

SEQ ID NO: 15 - DNA, 605 bp, Homo sapiens
  Encodes the transcipt CTD-2319I12.1-007 ENST00000588180.1
tcctcatcatcttcagcctagaggtacaagagcttcaggctgcaggagaccggcttttggatactgctaatcagtcctagcactctttctctttgaacctggacaaggtgtctgacccttctcaacacagcatcagacagccacctctattgactgaagttgacaatggagaataaaactcattacccctgctgaatccaggggcccctttttacagatgagaaaagttccagaaagacccagagacataatcaggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagca

SEQ ID NO: 16 - DNA, 466 bp, Homo sapiens
  Encodes the transcipt CTD-2319I12.1-008 ENST00000589777.1
tactgttttgagattatgtgaatgcattgcttttaactagaatatataagatgttaatgatattattcttcaagtaataaatcatattgcatattgaatgaaaagaaaaatgtagaactgacttcttaatcaagggctgttctccctagcatctgtcccaaaagtacggtggttttcagcacatccgttcttccataggtacctgcgtcgagctctgcacaggtgactgggactgcaaccccggagaccactgtgtcagcaatgggtgtggccatgagtgtgttgcagggtaaggacagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggt

SEQ ID NO: 17 - DNA, 2036 bp, Homo sapiens
  Encodes the transcipt LINC00330-001 ENST00000414562.1
aaacagttgagaacagactccaccttgtaggacagtggcatttctgaaagttcagaacaattccccgagcaactttacctcacaatctcattggaacggctgcatctgtggggccctgggagagactcagcagcagacagggtctccagagcacacgacaagaggaagaatgcatgaaggtccaccggcagtcgaatcagccaaagcacaagacaaacacaaagtcttgctctgtcgcccaggctggagtgcagtggtgcgaccttggctcactggtttggggcatgttcctgggtgtgtctgtgaggagcaagaaagccagttcaagtcccaaaacctcaaaagtagggaagccgacagggcagccttaagtctgtggctgaaggcctgagagaccctggcaaatcactgatataagtccaaaagctgaagaacttggaatctgatgttggaatctgatcttggcccatccttgaagaatctgggggtaataaataccctgaacattgatgaacccaaagggaagccagaaggtcagatgtcagctgtcgggaagaaaggctggataggaaataggaaaaagcagggcaaacaaggagcttcaaggactgaaacccttaagaaccacttgactctgtctctcaccacctccagccttgatgacacggtgacatatagaagaactgatgccctttgctttgagctgcacaaaaacctggcgctggacttggagaagctgaaagacaggacccggcaggagcctgaggagctgcaggctgctagcctgtagaccaccgtgctgtgttagccactgcagctgggaccattctgacactcagagcctggaaatggctgctccctcctgcctgctatccaagtatcctgtggatacttggcttttctcttgtgggcaacactaacctgaaactctttggggaagggaattgaggaaatgtagttccaagattagccaatttaactcagcataaacccatcacagggtttgactacagagttataacgttattctgtaggcaatgaaaacagaagtttttgaaaaaggcatcaatttgatgaaattgatattttggggaagattaatcttctgctgagtaaaatataacaggttggaggaagatgaaaatggagagagtagagtgtaattaaaagtttgttggaggccgggcacgggattacggctcactcctgtaatcccagcactttgggaggctgaggtgggcggatcacctgaggtcaggagcttgagatcatcccggccaacatagtgaaaccctgtctctaataaaaatacgaaaaattagctgggcatggtggcgcacacctgtaatctcagctactcgggaggctgaggcaggagaatcccttgaacccaggaggcagaggttgcaatgagccaagatggtgtgattgcactccagcccaggggacaatgtgagactctgtctcaaaaaaaaaaaaaaatttgttgtagtagtccaggtgtccgggtgttgttaccagggcacccatgatgaaataactgggagagtccttttttctgactggttttcttcagtccctttatctacaggcacaactgctgaagaaaccagatggcctgggatggcaccagagcttttttacccttgaccagatactagagggaattaagaccccacaagtgggcacgaactggaactggcgacccctagttgccttcagatcattaacacatcattataatgctaaaattccctgccattttgtgaacatgggttgcatgaagacgtaagtttatgaattgcctctgcacacctagagtcccaccctgtacaggctaacattcctccctacatgcacccccccacaaacacaccctgcctcccccagtccttagaaaccctatgcctggccgggcgcagtggctcacgcctgtaatcccagcactttgggaggctgaggcgggcggatcactaggtcaggagatcgagaccatcctggctaacacggtgaaaccctgtctttactaaaaatcc

SEQ ID NO: 18 - DNA, 742 bp, Homo sapiens
  Encodes the transcipt RP11-114H23.2-001 ENST00000548702.1
cctggcctccagcttctgtctgcttattcctgggatgttgttaagagcaccactcagcccctcagcccctaagcagcctcttccagtattagccagtgccccaggaaagatgtatcatctttcttgctgcccttgaagagtggtgtccagtgaatctttcaaagaatgatactgagcttcattgttgatgaacaaaaatgcaaatctcacccagagcatgcaggaacatgctataatcactacactcctagaatgtgatctctttcaatattgactgcatccatgccttccacacacttgcaccagttgggaggatggagatgtcatctgggcagagggagaagagcagtaccattcagaggtctgtccctcttaagatatattctctctgtcaacctagaccatagttgttcaacataatacttacacatttgaaaaaagaagattgttttattggagtgccttaagatgttttcaacatatggattttacagcagggcagttcaagaggcatgaggaatgtttcttttctctatttgttggagcactggagtgtgagctgaagcagcctaatcctgacttgcagaaatggagaggacatgacttaccggaattgttcatggaattggtttggctaattctcattgattctgccatttctccagaagccattactacagctgttgaaacaggggtagagaaaaatgacaggtgctaagtggtatctaggaggttaataatg

SEQ ID NO: 19 - DNA, 6675 bp, Homo sapiens; identical to SEQ ID NO: 4
  HERES gene